(19) United States
(12) Patent Application Publication
(10) Pub. No.: US 2014/0179713 A1
(43) Pub. Date: Jun. 26, 2014

(54) POLYMORPHIC FORM OF 5-(4-[4-(5-CYANO-1H-INDOL-3-YL) BUTYL] PIPERAZIN-1-YL) BENZOFURAN-2-CARBOXAMIDE AND PROCESS FOR PREPARING THEREOF

(71) Applicant: CADILA HEALTHCARE LIMITED, Ahmedabad (IN)

(72) Inventors: Shriprakash Dhar DWIVEDI, Ahmedabad (IN); Ramesh Chandra Singh, Ahmedabad (IN); Sachin Ashokrao Patil, Ahmedabad (IN)

(21) Appl. No.: 14/071,930

(22) Filed: Nov. 5, 2013

(30) Foreign Application Priority Data
Nov. 5, 2012 (IN) 3203/MUM/2012

(51) Int. Cl. C07D 405/12

(52) U.S. Cl. CPC C07D 405/12
USPC 514/254.09; 544/373

(57) ABSTRACT

The present invention provides a solid state Form-Z of 5-(4-[4-(5-cyano-1H-indol-3-yl)butyl]piperazin-1-yl)benzofuran-2-carboxamide. The present invention also provides a process for preparing Form-Z of 5-(4-[4-(5-cyano-1H-indol-3-yl)butyl]piperazin-1-yl)benzofuran-2-carboxamide comprising the steps of i) reacting solid state form of 5-(1-piperazinyl)benzofuran-2-carboxamide or its salts with 3-(4-chlorobutyl)-1H-indole-5-carbonitrile an organic solvent in presence of a base to obtain crude vilazodone free base; ii) purifying the crude vilazodone free base of step (i) in an organic solvent; iii) treating the purified vilazodone free base of step (ii) with an organic solvent to obtain solid state form-Z of vilazodone. The present invention further provides a pharmaceutical composition comprising a therapeutically effective amount of an amorphous form of vilazodone hydrochloride and use of solid state Form-Z of vilazodone for the treatment of major depressive disorders.

### FIELD OF THE INVENTION

The present invention relates to polymorphic form of 5-(4-[4-(5-cyano-1H-indol-3-yl)butyl]piperazin-1-yl)benzofuran-2-carboxamide. The present invention relates to a process for preparing polymorphic form of 5-(4-[4-(5-cyano-1H-indol-3-yl)butyl]piperazin-1-yl)benzofuran-2-carboxamide. In particular present invention relates to use of polymorphic form of 5-(4-[4-(5-cyano-1H-indol-3-yl)butyl]piperazin-1-yl)benzofuran-2-carboxamide in the preparation of its pharmaceutically acceptable salts thereof.

### BACKGROUND OF THE INVENTION

U.S. Pat. No. 5,532,241 discloses 5-(4-[4-(5-cyano-1H-indol-3-yl)butyl]piperazin-1-yl)benzo-furan-2-carboxamide i.e. vilazodone of Formula (1)

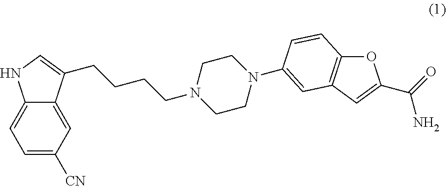 [SMILES: N#Cc1ccc2[nH]cc(CCCCN3CCN(c4ccc5oc(C(N)=O)cc5c4)CC3)c2c1]

or its pharmaceutically acceptable salt. Patent provides process for preparing vilazodone or its pharmaceutically acceptable salt thereof.

U.S. Pat. No. 5,977,112 relates to process for preparing intermediate involved in the preparation of vilazodone or its pharmaceutically acceptable salt thereof.

U.S. Pat. No. 6,509,475 B1 relates to process for preparing intermediate involved in the preparation of vilazodone or its pharmaceutically acceptable salt thereof.

U.S. Pat. No. 7,834,020 B2 claims vilazodone hydrochloride anhydrate in crystalline modification IV (Form IV) characterized by XRD. Patent discloses total 15 crystalline forms of vilazodone hydrochloride designated as crystalline Form-I to Form-XI and Form-XIII to Form-XVI.

U.S. Pat. No. 7,981,894 B2 claims vilazodone hydrochloride monohydrate in crystalline modification V (Form-V) having characteristic peaks in XRD.

### SUMMARY OF THE INVENTION

In one general aspect, there is provided a solid state form of 5-(4-[4-(5-cyano-1H-indol-3-yl)butyl]piperazin-1-yl)benzofuran-2-carboxamide i.e. vilazodone of Formula (1)

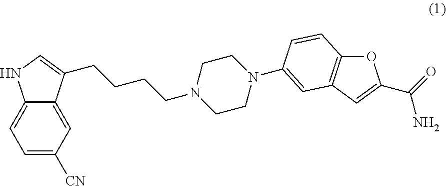 [SMILES: N#Cc1ccc2[nH]cc(CCCCN3CCN(c4ccc5oc(C(N)=O)cc5c4)CC3)c2c1]

The solid state form of 5-(4-[4-(5-cyano-1H-indol-3-yl)butyl]piperazin-1-yl)benzofuran-2-carboxamide i.e. vilazodone of Formula (1) has been designated as Form-Z.

In another aspect, there is provided a solid state Form-Z of 5-(4-[4-(5-cyano-1H-indol-3-yl)butyl]piperazin-1-yl)benzofuran-2-carboxamide i.e. vilazodone of Formula (1) characterized by XRD, DSC and TGA.

In another aspect, there is provided the use of solid state Form-Z of 5-(4-[4-(5-cyano-1H-indol-3-yl)butyl]piperazin-1-yl)benzofuran-2-carboxamide i.e. vilazodone of Formula (1) in the preparation of vilazodone hydrochloride.

In another aspect, there is provided a solid state form of hydrate or solvate of 5-(4-[4-(5-cyano-1H-indol-3-yl)butyl]piperazin-1-yl)benzofuran-2-carboxamide i.e. vilazodone of Formula (1).

In another aspect, there is provided a solid state form of solvate of 5-(4-[4-(5-cyano-1H-indol-3-yl)butyl]piperazin-1-yl)benzofuran-2-carboxamide i.e. vilazodone of Formula (1).

In another aspect, there is provided a solid state Form-Z of vilazodone of Formula (1) having a total purity of greater than about 99%, specifically greater than about 99.5%, more specifically greater than about 99.9%, and most specifically greater than about 99.98% as measured by HPLC.

In another aspect, there is provided a process for preparing a Form-Z of 5-(4-[4-(5-cyano-1H-indol-3-yl)butyl]piperazin-1-yl)benzofuran-2-carboxamide i.e. vilazodone of Formula (1).

In another aspect, there is provided the use of solid state Form-Z of 5-(4-[4-(5-cyano-1H-indol-3-yl)butyl]piperazin-1-yl)benzofuran-2-carboxamide i.e. vilazodone of Formula (1)

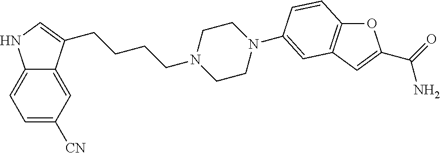 [SMILES: N#Cc1ccc2[nH]cc(CCCCN3CCN(c4ccc5oc(C(N)=O)cc5c4)CC3)c2c1]

in the preparation of vilazodone hydrochloride of Formula (2)

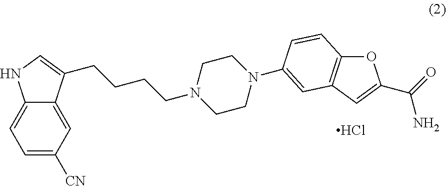 [SMILES: N#Cc1ccc2[nH]cc(CCCCN3CCN(c4ccc5oc(C(N)=O)cc5c4)CC3)c2c1 ; Cl]

### BRIEF DESCRIPTION OF THE ACCOMPANYING DRAWINGS

FIG. 1: Shows X-ray diffractogram (XRD) of Form-Z of vilazodone of Formula (1).

FIG. 2: Shows differential scanning Calorimetry (DSC) of Form-Z of vilazodone of Formula (1).

### DETAILED DESCRIPTION OF THE INVENTION

An embodiment of the present invention provides a solid state form of 5-(4-[4-(5-cyano-1H-indol-3-yl)butyl]piperazin-1-yl)benzofuran-2-carboxamide i.e. vilazodone of Formula (1)

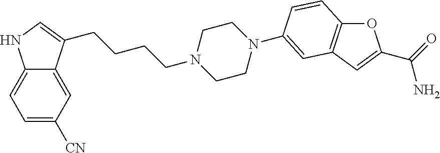 [SMILES: N#Cc1ccc2[nH]cc(CCCCN3CCN(c4ccc5oc(C(N)=O)cc5c4)CC3)c2c1]

The present invention has designated the solid state form of 5-(4-[4-(5-cyano-1H-indol-3-yl)butyl]piperazin-1-yl)benzofuran-2-carboxamide i.e. vilazodone of Formula (1) as Form-Z and the term Form-Z will be used for describing the said form of vilazodone of Formula (1).

A solid state Form-Z of 5-(4-[4-(5-cyano-1H-indol-3-yl)butyl]piperazin-1-yl)benzofuran-2-carboxamide i.e. vilazodone of Formula (1) is characterized by X-ray powder diffraction having characteristic peaks at about 5.5, 11.0, 15.9, 18.7, 20.7, 22.5, 25.0, 26.1, and 27.0±0.2 degree 2θ.

A solid state Form-Z of 5-(4-[4-(5-cyano-1H-indol-3-yl)butyl]piperazin-1-yl)benzofuran-2-carboxamide i.e. vilazodone of Formula (1) is further characterized by X-ray powder diffraction having characteristic peaks at about 7.5, 14.2, 15.2, 16.7, 17.7 28.6, 29.8, 30.8 and 32.2±0.2 degree 2θ.

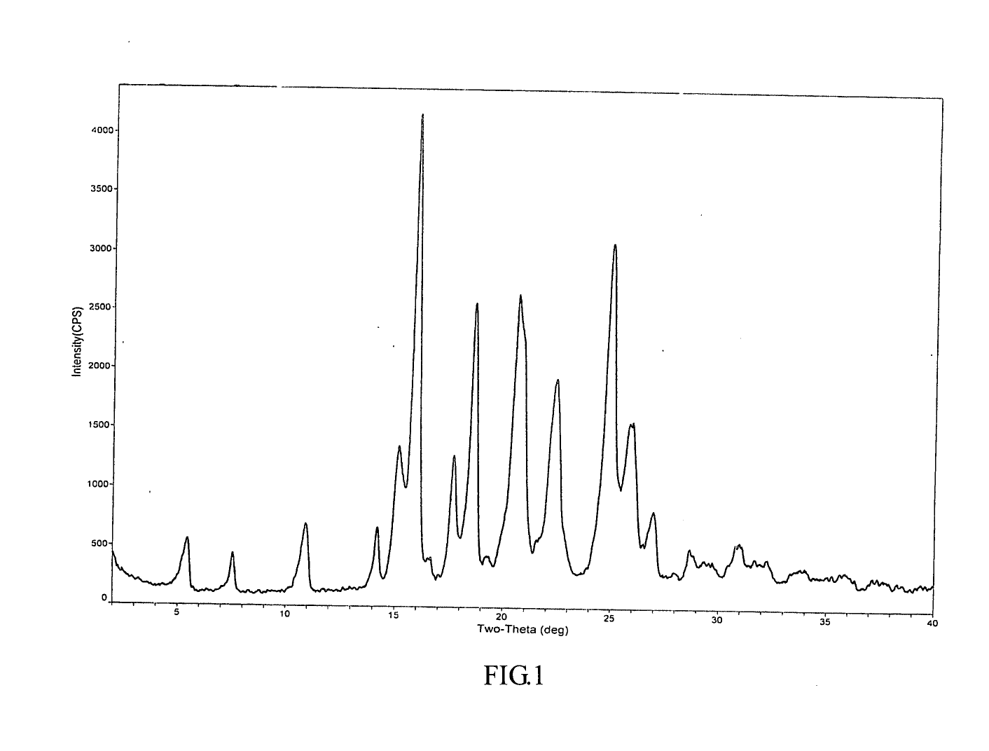

A solid state Form-Z of 5-(4-[4-(5-cyano-1H-indol-3-yl)butyl]piperazin-1-yl)benzofuran-2-carboxamide i.e. vilazodone of Formula (1) is characterized by X-ray powder diffraction as depicted in FIG. 1.

A solid state Form-Z of 5-(4-[4-(5-cyano-1H-indol-3-yl)butyl]piperazin-1-yl)benzofuran-2-carboxamide i.e. vilazodone of Formula (1) is characterized by Differential Scanning Calorimetry (DSC) having two endotherms at about 88° C. and 210° C. respectively.

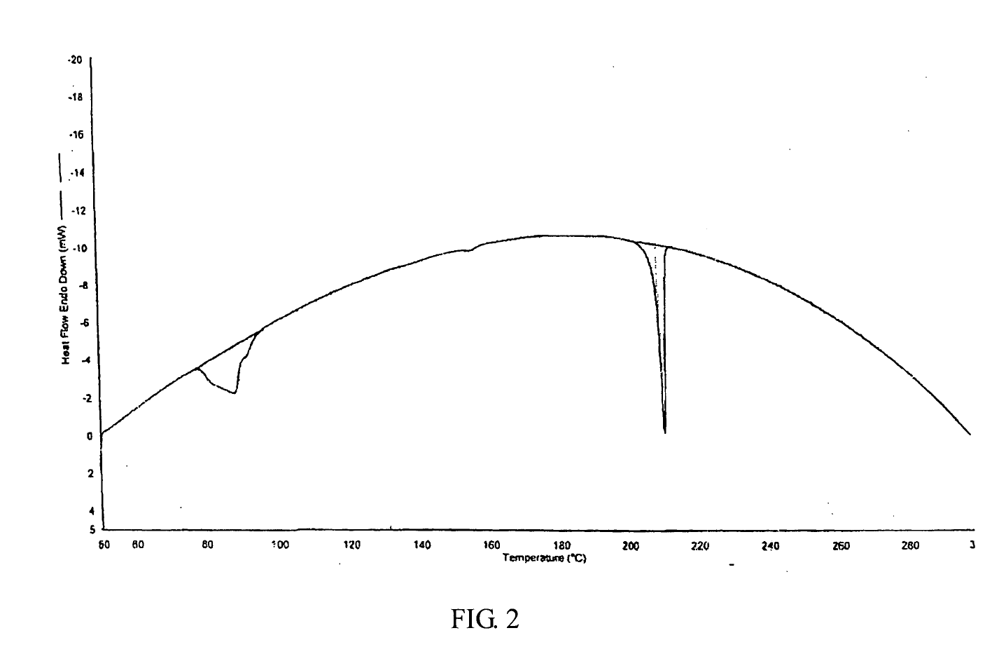

A solid state Form-Z of 5-(4-[4-(5-cyano-1H-indol-3-yl)butyl]piperazin-1-yl)benzofuran-2-carboxamide i.e. vilazodone of Formula (1) is characterized by Differential Scanning Calorimetry as depicted in FIG. 2.

A solid state Form-Z of 5-(4-[4-(5-cyano-1H-indol-3-yl)butyl]piperazin-1-yl)benzofuran-2-carboxamide i.e. vilazodone of Formula (1) is having water content of about 4%.

A solid state Form-Z of 5-(4-[4-(5-cyano-1H-indol-3-yl)butyl]piperazin-1-yl)benzofuran-2-carboxamide i.e. vilazodone of Formula (1) is a monohydrate form.

A solid state Form-Z of 5-(4-[4-(5-cyano-1H-indol-3-yl)butyl]piperazin-1-yl)benzofuran-2-carboxamide i.e. vilazodone of Formula (1) is substantially free from residual organic solvents.

In another aspect, there is provided use of solid state Form-Z of 5-(4-[4-(5-cyano-1H-indol-3-yl)butyl]piperazin-1-yl)benzofuran-2-carboxamide i.e. vilazodone of Formula (1) in the preparation of vilazodone hydrochloride.

In another aspect, there is provided solid state form of hydrate or solvate of 5-(4-[4-(5-cyano-1H-indol-3-yl)butyl]piperazin-1-yl)benzofuran-2-carboxamide i.e. vilazodone of Formula (1).

In another aspect, there is provided solid state form of solvate of 5-(4-[4-(5-cyano-1H-indol-3-yl)butyl]piperazin-1-yl)benzofuran-2-carboxamide i.e. vilazodone of Formula (1).

In another aspect, there is provided a new process for the preparation of 5-(1-piperazinyl)benzofuran-2-carboxamide of Formula (G), in the preparation of vilazodone of Formula (1)

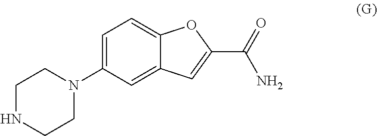 [SMILES: NC(=O)c1cc2cc(N3CCNCC3)ccc2o1]

the process comprising:

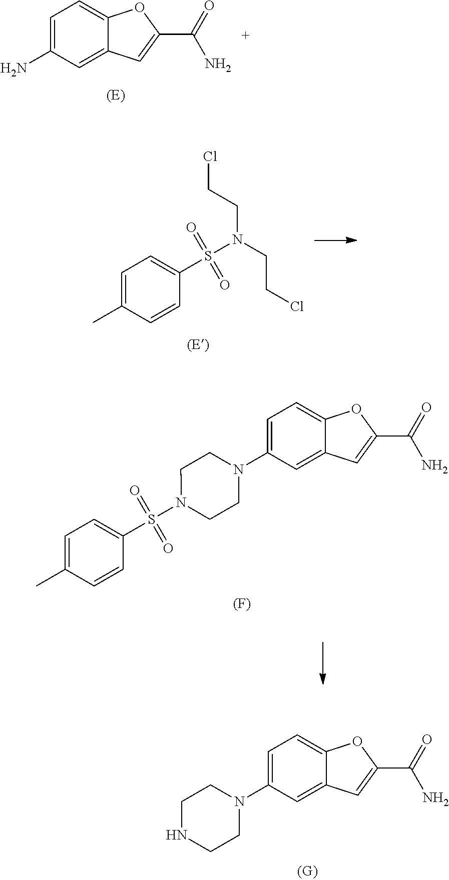

i) reacting 5-Aminobenzofuran-2-carboxamide of Formula (E) with N,N-bis(2-chloroethyl)-4-methylbenzenesulfonamide of Formula (E′) in presence of solvent and base to obtain 5-(4-tosylpiperazin-1-yl)benzofuran-2-carboxamide of Formula (F);

ii) deprotecting 5-(4-tosylpiperazin-1-yl)benzofuran-2-carboxamide of Formula (F) by reaction with a suitable acid or its hydrohalide salt and p-Hydroxy benzoic acid; and

iii) isolating 5-(1-piperazinyl)benzofuran-2-carboxamide of Formula (G) in presence of a base.

The suitable solvent for step (i) comprises one or more of hydrocarbons, nitriles, amides, alcohol, ketones, ester and the like. In particular, the suitable solvent comprises toluene, xylene, ethylbenzene dimethyl formamide, dimethyl acetamide, acetonitrile, C₁-C₄ straight chain or branched alcohols, acetone, methyl isobutyl ketone, methyl ethyl ketone, ethyl acetate, isopropyl acetate, butyl acetate and the likes. Particularly, the solvent may be methanol.

The suitable base for step (i) comprises of an alkali and alkaline metal hydroxide and carbonate, in particular the suitable alkali metal hydroxide comprises of sodium hydroxide, potassium hydroxide, lithium hydroxide, and the like, carbonate comprises of sodium carbonate, potassium carbonate, cesium carbonate and the like. Particularly, the base may be Diisopropylethylamine.

The suitable solvent used in step (ii) for removal of amino group protection from 5-(4-tosylpiperazin-1-yl)benzofuran-2-carboxamide of Formula (F) is selected from one or more of hydrocarbons, nitriles, amides, alcohol, ketones, ester and the like. In particular, the suitable solvent comprises toluene, xylene, ethylbenzene dimethyl formamide, dimethyl acetamide, acetonitrile, C₁-C₄ straight chain or branched alcohols, acetone, methyl isobutyl ketone, methyl ethyl ketone, ethyl acetate, isopropyl acetate, butyl acetate and the likes. Particularly, the solvent may be water and ethyl acetate.

The suitable acid in step (ii) may be selected from hydrochloric acid, hydrobromic acid, hydroiodic acid, acetic acid, benzoic acid, p-hydroxy benzoic acid and like or a mixture thereof. More particularly the acid used is a mixture of hydrobromic acid, acetic acid as well as p-hydroxy benzoic acid.

The suitable solvent for step (iii) for isolating a solid state form of 5-(1-piperazinyl)benzofuran-2-carboxamide of Formula (G) comprises one or more of hydrocarbons, water, nitriles, amides, alcohol, ketones, ester and the like. In particular, the suitable solvent comprises toluene, xylene, ethylbenzene dimethyl formamide, dimethyl acetamide, acetonitrile, C₁-C₄ straight chain or branched alcohols, acetone, methyl isobutyl ketone, methyl ethyl ketone, ethyl acetate, isopropyl acetate, butyl acetate and the likes. Particularly, the solvent may be ethyl acetate.

The suitable base employed in step (iii) comprises of an alkali and alkaline metal hydroxide and carbonate, in particular the suitable alkali metal hydroxide comprises of sodium hydroxide, potassium hydroxide, lithium hydroxide, and the like, carbonate comprises of sodium carbonate, potassium carbonate, cesium carbonate and the like. Particularly, the base may be sodium hydroxide.

In another aspect, there is provided a process for preparing a solid state form-Z of 5-(4-[4-(5-cyano-1H-indol-3-yl)butyl]piperazin-1-yl)benzofuran-2-carboxamide i.e. vilazodone of Formula (1)

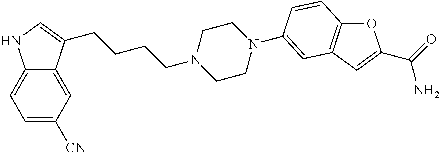 [SMILES: N#Cc1ccc2[nH]cc(CCCCN3CCN(c4ccc5oc(C(N)=O)cc5c4)CC3)c2c1]

the process comprising:

i) reacting solid state form of 5-(1-piperazinyl)benzofuran-2-carboxamide Formula (G) or its salts with 3-(4-chlorobutyl)-1H-indole-5-carbonitrile of Formula (H) in an organic solvent in presence of a base to obtain crude vilazodone free base;

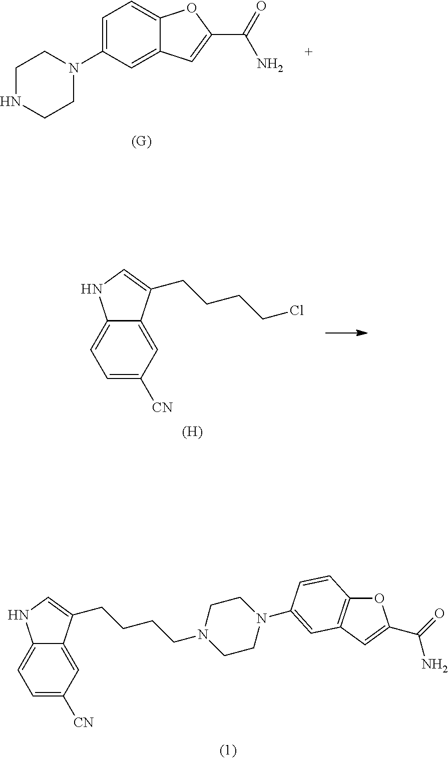

ii) purifying the crude vilazodone free base of step (i) in an organic solvent;

iii) treating the purified vilazodone free base of step (ii) with an acid and an organic solvent to obtain solid state form-Z of vilazodone of Formula (1).

The suitable solvent for step (i) comprises one or more of solvent comprises of hydrocarbons, nitriles, amides, alcohol, ketones, ester and the like. In particular, the suitable solvent comprises toluene, xylene, ethylbenzene dimethyl formamide, dimethyl sulfoxide, dimethyl acetamide, acetonitrile, C₁-C₄ straight chain or branched alcohols, acetone, methyl isobutyl ketone, methyl ethyl ketone, ethyl acetate, isopropyl acetate and butyl acetate and the like. Particularly, the solvent may be dimethyl sulfoxide.

The suitable base for step (i) comprises of an organic or inorganic base; an organic base may be selected from diisopropylethylamine, diisopropylamine, trimethylamine, diethylamine, piperidine, morpholine, pyridine, DBU, DABCO and the like; the inorganic base comprises of an alkali and alkaline metal hydroxide and carbonate, in particular the suitable alkali metal hydroxide comprises of sodium hydroxide, potassium hydroxide, lithium hydroxide, and the like, carbonate comprises of sodium carbonate, potassium carbonate, cesium carbonate and the like. Particularly, the base may be diisopropylethylamine.

The obtained crude vilazodone of step (i) is purified in step (ii) using one or more of solvent comprises of hydrocarbons, nitriles, amides, alcohol, ketones, ester and the like. In particular, the suitable solvent comprises toluene, xylene, ethylbenzene dimethyl formamide, dimethyl sulfoxide, dimethyl acetamide, acetonitrile, C₁-C₄ straight chain or branched alcohols, acetone, methyl isobutyl ketone, methyl ethyl ketone, ethyl acetate, isopropyl acetate and butyl acetate and the like. Particularly, the solvent may be acetone.

The suitable acid for step (iii) may be selected from both organic and mineral acid, preferably acetic acid.

The suitable solvent for step (iii) comprises one or more of solvent comprises of hydrocarbons, nitriles, amides, alcohol, ketones, ester and the like. In particular, the suitable solvent comprises toluene, xylene, ethylbenzene dimethyl formamide, dimethyl acetamide, acetonitrile, C₁-C₄ straight chain or branched alcohols, acetone, methyl isobutyl ketone, methyl ethyl ketone, ethyl acetate, isopropyl acetate and butyl acetate and the like. Particularly, the solvent may be methanol.

In another aspect, there is provided a new process for preparing vilazodone of Formula (1) or its pharmaceutically acceptable salts thereof;

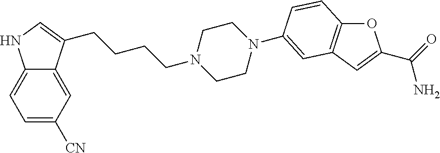 [SMILES: N#Cc1ccc2[nH]cc(CCCCN3CCN(c4ccc5oc(C(N)=O)cc5c4)CC3)c2c1]

the process comprising:

i) reacting 5-Aminobenzofuran-2-carboxamide of Formula (E) with N,N-bis(2-chloroethyl)-4-methylbenzenesulfonamide of Formula (E′) in an organic solvent in presence of a base to obtain 5-(4-tosylpiperazin-1-yl)benzofuran-2-carboxamide of Formula (F);

ii) deprotecting 5-(4-tosylpiperazin-1-yl)benzofuran-2-carboxamide of Formula (F) in the presence of acid;

iii) isolating a solid state form of 5-(1-piperazinyl)benzofuran-2-carboxamide of Formula (G) in an organic solvent in presence of a base;

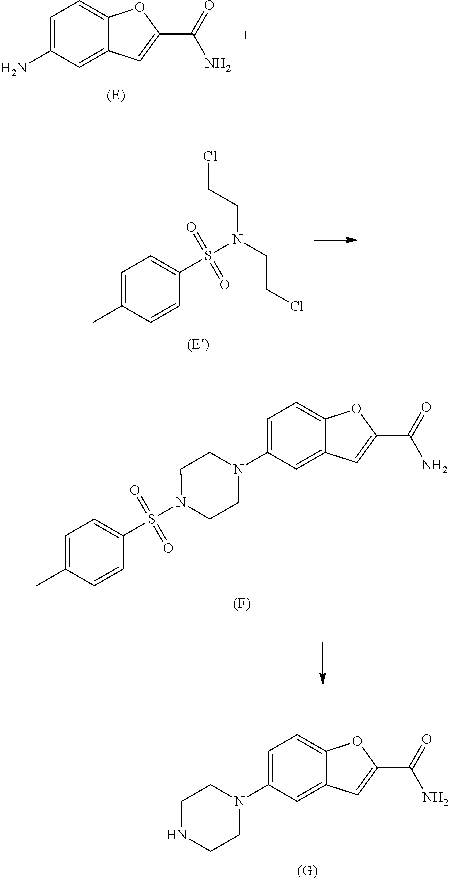

iv) reacting 5-(1-piperazinyl)benzofuran-2-carboxamide of Formula (G) with 3-(4-chlorobutyl)-1H-indole-5-carbonitrile of Formula (H) in an organic solvent in presence of a base to obtain vilazodone of Formula (1);

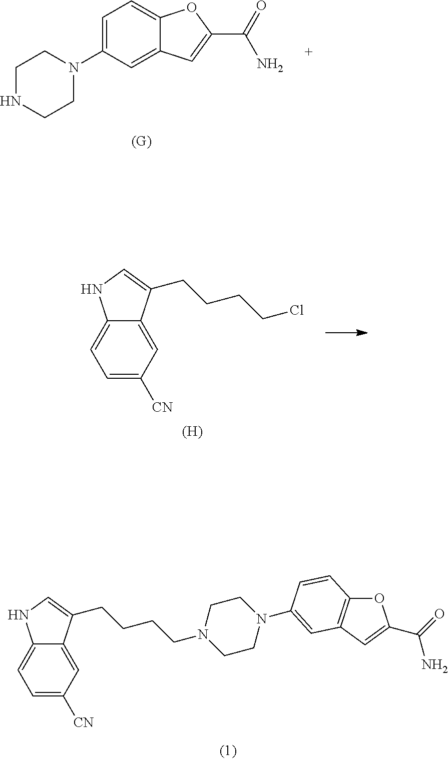

v) optionally converting vilazodone of Formula (1) into its pharmaceutically acceptable salts thereof.

The suitable solvent for step (i) comprises one or more of hydrocarbons, nitriles, amides, alcohol, ketones, ester and the like. In particular, the suitable solvent comprises toluene, xylene, ethylbenzene dimethyl formamide, dimethyl acetamide, acetonitrile, C₁-C₄ straight chain or branched alcohols, acetone, methyl isobutyl ketone, methyl ethyl ketone, ethyl acetate, isopropyl acetate, butyl acetate and the likes. Particularly, the solvent may be methanol.

The suitable base for step (i) comprises of an alkali and alkaline metal hydroxide and carbonate, in particular the suitable alkali metal hydroxide comprises of sodium hydroxide, potassium hydroxide, lithium hydroxide, and the like, carbonate comprises of sodium carbonate, potassium carbonate, cesium carbonate and the like. Particularly, the base may be Diisopropylethylamine.

The suitable solvent used in step (ii) for removal of amino group protection from 5-(4-tosylpiperazin-1-yl)benzofuran-2-carboxamide of Formula (F) is selected from one or more of hydrocarbons, nitriles, amides, alcohol, ketones, ester and the like. In particular, the suitable solvent comprises toluene, xylene, ethylbenzene dimethyl formamide, dimethyl acetamide, acetonitrile, C₁-C₄ straight chain or branched alcohols, acetone, methyl isobutyl ketone, methyl ethyl ketone, ethyl acetate, isopropyl acetate, butyl acetate and the likes. Particularly, the solvent may be water and ethyl acetate.

The suitable acid in step (ii) may be selected from hydrochloric acid, hydrobromic acid, hydroiodic acid, acetic acid, benzoic acid, p-hydroxy benzoic acid and like or a mixture thereof. More particularly the acid used is a mixture of Hydrobromic acid, acetic acid as well as p-Hydroxy benzoic acid.

The suitable solvent for step (iii) for isolating a solid state form of 5-(1-piperazinyl)benzofuran-2-carboxamide of Formula (G) comprises one or more of hydrocarbons, water, nitriles, amides, alcohol, ketones, ester and the like. In particular, the suitable solvent comprises toluene, xylene, ethylbenzene dimethyl formamide, dimethyl acetamide, acetonitrile, C₁-C₄ straight chain or branched alcohols, acetone, methyl isobutyl ketone, methyl ethyl ketone, ethyl acetate, isopropyl acetate, butyl acetate and the likes. Particularly, the solvent may be ethyl acetate.

The suitable base for step (iii) comprises of an alkali and alkaline metal hydroxide and carbonate, in particular the suitable alkali metal hydroxide comprises of sodium hydroxide, potassium hydroxide, lithium hydroxide, and the like, carbonate comprises of sodium carbonate, potassium carbonate, cesium carbonate and the like. Particularly, the base may be sodium hydroxide.

The suitable solvent for step (iv) comprises one or more of solvent comprises of hydrocarbons, nitriles, amides, alcohol, ketones, ester and the like. In particular, the suitable solvent comprises toluene, xylene, ethylbenzene dimethyl formamide, dimethyl sulfoxide, dimethyl acetamide, acetonitrile, C₁-C₄ straight chain or branched alcohols, acetone, methyl isobutyl ketone, methyl ethyl ketone, ethyl acetate, isopropyl acetate and butyl acetate and the like. Particularly, the solvent may be dimethyl sulfoxide.

The suitable base for step (iv) comprises of an organic or inorganic base; an organic base may be selected from diisopropylethylamine, diisopropylamine, trimethylamine, diethylamine, piperidine, morpholine, pyridine, DBU, DABCO and the like; the inorganic base comprises of an alkali and alkaline metal hydroxide and carbonate, in particular the suitable alkali metal hydroxide comprises of sodium hydroxide, potassium hydroxide, lithium hydroxide, and the like, carbonate comprises of sodium carbonate, potassium carbonate, cesium carbonate and the like. Particularly, the base may be diisopropylethylamine.

The suitable solvent for step (v) comprises one or more of solvent comprises of hydrocarbons, nitriles, amides, alcohol, ketones, ester and the like. In particular, the suitable solvent comprises toluene, xylene, ethylbenzene dimethyl formamide, dimethyl sulfoxide, dimethyl acetamide, acetonitrile, C₁-C₄ straight chain or branched alcohols, acetone, methyl isobutyl ketone, methyl ethyl ketone, ethyl acetate, isopropyl acetate and butyl acetate and the like. Particularly, the solvent may be acetone.

In another aspect, there is provided vilazodone hydrochloride having particle size in terms of d95 is less than about 100 microns.

In another aspect, there is provided vilazodone hydrochloride having particle size in terms of d95 is less than about 10 microns

In another aspect, there is provided a process for preparing vilazodone hydrochloride having particle size in terms of d95 is less than about 10 microns; to the process comprising:

i) milling vilazodone hydrochloride having particle size in terms of d95 is greater than about 100 microns;

ii) slurrying micronized vilazodone hydrochloride in one or more organic solvents to form a solution;

iii) isolating vilazodone hydrochloride having particle size in terms of d95 is less than about 10 microns.

In another aspect, there is provided vilazodone of Formula (1) or its pharmaceutically acceptable salts thereof having a total purity of greater than about 99%, specifically greater than about 99.5%, more specifically greater than about 99.9%, and most specifically greater than about 99.97% as measured by HPLC.

In another aspect, there is provided a pharmaceutical composition comprising as its active ingredient of vilazodone of Formula (1). With the active ingredient, the pharmaceutical composition includes one or more pharmaceutically acceptable excipients/diluents. The pharmaceutical composition of the present invention may be in the form of a solid or liquid dosage forms for oral, parenteral or topical use and may have immediate or sustained release characteristics. The dosage forms possible include tablets, capsules, powders, granules, creams, lotions, ointments, injectables, ophthalmic or optic solutions, suspensions, elixirs and the like.

According to the present invention, the process for the preparation of vilazodone of Formula (1) can be illustrated by below mentioned Scheme-1, which should not be considered as limiting the scope of the invention.

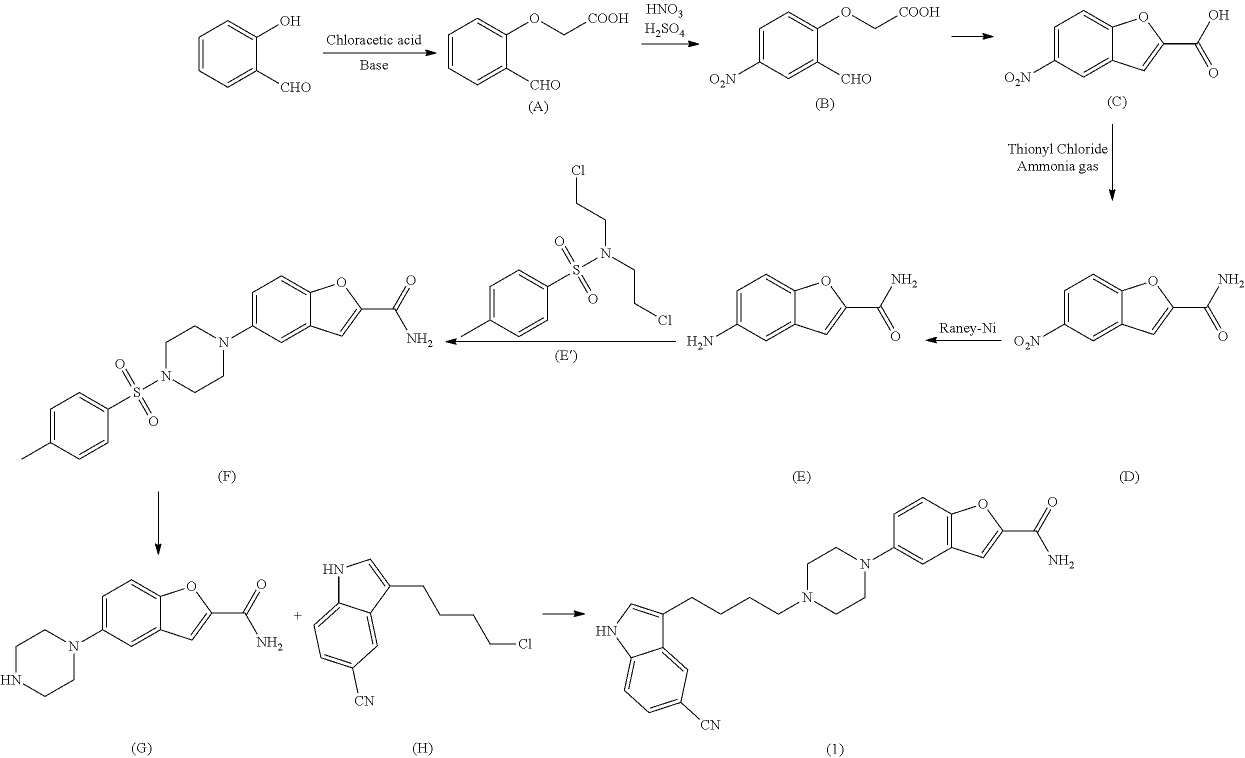

### EXAMPLES

While the present invention is described with respect to particular examples and preferred embodiments, it is understood that the present invention is not limited to these examples and embodiments. The present invention, therefore, includes variations form the particular examples embodiments described herein, as will be applicant to one of skill in the art.

### Example 1

### Preparation of 2-(2-formylphenoxy)acetic acid of Formula (A)

Water (700 ml), salicyaldehyde (100 g), chloroacetic acid (77 g) and sodium hydroxide solution (165 ml) were added to a four-neck two litre round bottom flask at 30° C. The reaction mass was heated to 100° C. and maintained for 3 hours. Conc.HCl (100 ml) was added to adjust pH 1 to 2. The reaction mass was cooled to 30° C. and further cooled to 15° C. and stirred for 1 hour. The product was filtered and washed with chilled water (2×25 ml) to afford 2-(2-formylphenoxy)acetic acid of Formula (A).

### Example 2

### Preparation of 2-(2-formyl-4-nitrophenoxy)acetic acid of Formula (B)

Sulfuric acid (550 ml) added to a four-neck two litre round bottom flask at 35° C. and cooled to 5° C. and added lot wise 2-(2-formylphenoxy)acetic acid of Formula (A) (100 g). The reaction mass was stirred for 15 minutes. In another two litre round bottom flask nitric acid (45 ml) was added at 35° C. The reaction mass was cooled to 15° C. followed by addition of sulfuric acid (50 ml) and stirred for 15 minutes. The reaction mass of second round bottom flask was added into first round bottom flask at 5° C. and stirred for 1 hour below 15° C. In another round bottom flask water (1500 ml) was added at 35° C. and cooled to 10° C. The reaction mass of first round bottom flask was dumped into third round bottom flask having water and stirred for 1 hour. Finally the product was washed chilled water (2×100 ml) at 35° C. to afford 2-(2-formyl-4-nitrophenoxy)acetic acid of Formula (B).

### Example 3

### Preparation of 5-nitrobenzofuran-2-carboxylic acid of Formula (C)

Acetic anhydride (500 ml) and 2-(2-formyl-4-nitrophenoxy) acetic acid of Formula (B) (100 g) were added to a four-neck two litre round bottom flask at 35° C. and stirred for 15 minutes. Anhydrous Sodium acetate (150 g) was added to the reaction mass and heated to 125° C. and maintained for 8 hours. The reaction mass was cooled to 100° C. and further cooled to 80° C. and toluene (200 ml) was added. The reaction mass was stirred for 30 minutes and under vacuum up to 85° C. and cooled to 35 35° C. Water (500 ml) was added and conc. HCl (200 ml) was added to adjust pH 1-2. The reaction mass was stirred for 60 minutes. The product was washed with water (500 ml) and stirred for 30 minutes at 35° C. The product was dried for 16 hours at 65° C. Toluene (200 ml) was added to the product at 35° C. and heated to 90° C. and maintained for 30 minutes and cooled to 35° C., further cooled to 5° C. and maintained for 30 minutes. The product was washed with chilled toluene (2×25 ml) to afford 5-nitrobenzofuran-2-carboxylic acid of Formula (C).

### Example 4

### Preparation of 5-nitrobenzofuran-2-carboxamide of Formula (D)

Toluene (1200 ml) and 5-nitrobenzofuran-2-carboxylic acid of Formula (C) were added to a four-neck two litre round bottom flask at 35° C. and stirred for 15 minutes. DMF (10 ml) was added and heated to 70° C. followed by addition of Thionyl chloride (86.4 g) and heated to 115° C. and maintained for 30 minutes. Toluene was distilled out and reaction mass was cooled to 35° C. and further cooled to 10° C. Ammonia gas was passed till pH was 8 to 9. The product was washed with Toluene (2×100 ml) and dried for 60 minutes and dried in hot air oven at for 12.0 hrs at 65° C. To the obtained product DMF (250 ml) was added at 35° C. and heated to 130° C. The reaction mass was stirred for 30 minutes and cooled to 95° C. followed by addition of water (2300 ml) and stirred for 30 minutes. The reaction mass was cooled to 50° C. The product was filtered and washed with water (2×100 ml) to afford 5-nitrobenzofuran-2-carboxamide of Formula (D).

### Example 5

### Preparation of 5-aminobenzofuran-2-carboxamide of Formula (E)

In a 5.0 lit Autoclave methanol (2000 ml) and 5-nitrobenzofuran-2-carboxamide of Formula (D) (100 g) and Raney Nickel (40.0 g) were added at 35° C. The reaction mass was flushed with two times Nitrogen pressure (0.5-1.0 Kg) and released. Hydrogen pressure up to 5.0 Kg/cm² was applied at 35° C. The reaction mass was heated to 60° C. and maintained at 6.0 Kg/cm² pressure for 14 hours at 60° C. The reaction mass was cooled to 35° C. and Hydrogen pressure was released. Organic layer was distilled out under vacuum at 60° C. Hexane (50 ml) was added and stirred for 30 minutes and reaction was cooled to 35° C. The product was washed with hexane (2×25 ml) to afford 5-aminobenzofuran-2-carboxamide of Formula (E).

### Example 6

### Preparation of N,N-bis(2-chloroethyl)-4-methylbenzenesulfonamide (E′)

To a reaction flask, 100 g. of bis(2-chloroethyl)amine hydrochloride and 500 ml. of MDC were added and stirred. To the reaction mass was added triethyl amine as a base and again stirred for 15 minutes at room temperature. p-Toluene sulfonylchloride solution (in MDC) was added to the reaction mass and heated to 40° C. and maintained for 6 hours. TLC was checked for completion of reaction and treated with 250 ml of water and organic layer was separated. To this reaction mass, was added 250 ml of 10% HCl solution and stirred. The organic layer was separated, treated with sodium sulfate and washed thrice with MDC as the solvent was removed under vacuum. To this mass hexane was added and again removed under vacuum twice. Once again Hexane was added and reaction mass stirred for two hours and cooled which afforded the product N,N-bis(2-chloroethyl)-4-methylbenzene sulfonamide which was filtered, washed with hexane and dried.

### Example 7

### Preparation of 5-(4-tosylpiperazin-1-yl)benzofuran-2-carboxamide (F)

1200 ml DIPEA as a base and 5-aminobenzofuran-2-carboxamide (E) were added to a reaction flask and stirred for 15 minutes to which 252 g of N,N-bis(2-chloroethyl-4-methylbenzenesulfonamide was added, stirred, heated at 120° C. and maintained for 10 hours. The completion of reaction was checked by TLC and cooled after which DIPEA was decanted and 1000 ml of Methanol was added to it. The mass was again heated to about 55° C. and stirred for 1 hour after which it was cooled to afford us with 5-(4-tosylpiperazin-1-yl)benzofuran-2-carboxamide (F) which was filtered under vacuum, and washed with methanol and dried.

### Example 8

### Preparation of 5-(1-piperazinyl)benzofuran-2-carboxamide of Formula (G)

To a mixture of 500 ml of hydrobromic acid in acetic acid was added portion wise 169 g of p-hydroxy benzoic acid and stirred. To this mixture was added portion wise 100 g of 5-(4-tosylpiperazin-1-yl)benzofuran-2-carboxamide (F) and stirred for some time after which the temperature was raised to around 50° C. and the mass was stirred for 3 hours. After the completion of reaction was checked by TLC, it was slowly cooled at around 10° C. and 1.5 liters of cold water was added to it. The solid obtained was filtered and washed with Ethyl Acetate four times and the aqueous layer was then collected and the pH was adjusted to 10 by using sodium hydroxide solution. The mass was stirred for half an hour and the solid thus obtained was filtered, washed with water and dried to afford us with 5-(1-piperazinyl)benzofuran-2-carboxamide of Formula (G)

### Example 9

### Preparation of Form-Z of Vilazodone of Formula (1)

DMSO (1000 ml), 5-(1-piperazinyl)benzofuran-2-carboxamide of Formula (G) (100 g), 3-(4-chlorobutyl)-1H-indole-5-carbonitrile of Formula (H) and diisopropylethylamine (DIPEA) (1500 ml) were added to a four-neck two litre round bottom flask at 35° C. and reaction mass was heated to 80° C. and maintained for 30 hours. The reaction mass was cooled to 55° C. The reaction mass was treated with EDTA (3 g) and charcoal (10 g). The residue was filtered and treated with DMSO (800 ml) at 80-85° C. and stirred for 15 minutes. The reaction mass was treated with acetone at 20-25° C. and stirred for 1 hour. The reaction mass was washed with acetone (2×100) and treated with acetic acid (720 ml) and methanol (500 ml) at 25-30° C. and stirred for 10 minutes. The reaction mass is heated to 80-81° C. and treated with activated charcoal (10 g) and stirred for 15-20 min. The reaction mass was cooled to 15-20° C., filtered and washed with methanol (2×100 ml). The product was dried in hot air oven at 60-65° C. for 12-14 hours. The product was further dried in oven at 105° C. for 4 hours afforded Form-Z of vilazodone of Formula (1). (HPLC purity >99.70%).

XRD and DSC pattern is substantially as depicted in FIG. 1 and FIG. 2 respectively.

### Example 10

### Preparation of Vilazodone Hydrochloride

Form-Z of vilazodone free base of Formula (1) (60 g) and acetone (1200 ml) were heated to 50-55° C. An another assembly was arranged in a tub using diisopropyl ether (1000 ml) and conc. hydrochloric acid (16 ml) at 0-5° C. First reaction mass was added into second reaction mass and stirred for 30 minutes. The reaction mass was filtered and washed with chilled DIPE (120 ml) at 0-5° C. afforded vilazodone hydrochloride. (HPLC purity>99.80%).

### Example 11

### Preparation of Vilazodone Hydrochloride

Form-Z of vilazodone free base of Formula (1) (60 g) and acetone (1200 ml) were heated to 50-55° C. An another assembly was arranged in a tub using acetone (300 ml) and conc. hydrochloric acid (16 ml) at 0-5° C. First reaction mass was added into second reaction mass and stirred for 30 minutes. The reaction mass was filtered and washed with chilled acetone (120 ml) at 0-5° C. afforded vilazodone hydrochloride. (HPLC purity>99.85%).

We claim:

1. A solid state Form-Z of 5-(4-[4-(5-cyano-1H-indol-3-yl)butyl]piperazin-1-yl)benzofuran-2-carboxamide i.e. vilazodone of Formula (1) 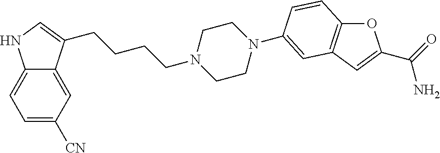 [SMILES: N#Cc1ccc2[nH]cc(CCCCN3CCN(c4ccc5oc(C(N)=O)cc5c4)CC3)c2c1]
which is characterized by one or more of the following properties:
i) a powder X-ray diffraction pattern having characteristic peaks expressed in degrees 2θ at about 5.5, 11.0, 15.9, 18.7, 20.7, 22.5, 25.0, 26.1, and 27.0±0.2 degree 2θ substantially as depicted in FIG. 1.
ii) a powder X-ray diffraction pattern having additional peaks at about 7.5, 14.2, 15.2, 16.7, 17.7 28.6, 29.8, 30.8 and 32.2±0.2 degree 2θ substantially as depicted in FIG. 1.
iii) a Differential Scanning Calorimetry (DSC) having two endotherms at about 88° C. and 210° C. substantially as depicted in FIG. 2.

2. The solid state Form-Z of vilazodone as claimed in claim 1, having water content of about 4% wt/wt.

3. The solid state Form-Z of vilazodone as claimed in claim 2 is a monohydrate form.

4. The solid state Form-Z of vilazodone as claimed in claim 1, which is substantially free from residual organic solvents.

5. The solid state Form-Z of vilazodone as claimed in claim 1 having a particle size in terms of d95 is less than about 100 microns.

6. A process for preparing a solid state form-Z of 5-(4-[4-(5-cyano-1H-indol-3-yl)butyl]piperazin-1-yl)benzofuran-2-carboxamide i.e. vilazodone of Formula (1) 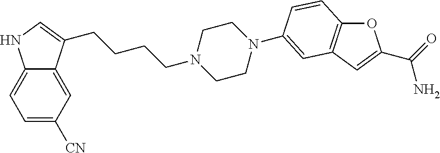 [SMILES: N#Cc1ccc2[nH]cc(CCCCN3CCN(c4ccc5oc(C(N)=O)cc5c4)CC3)c2c1]
the process comprising:
i) reacting solid state form of 5-(1-piperazinyl)benzofuran-2-carboxamide Formula (F) or its salts with 3-(4-chlorobutyl)-1H-indole-5-carbonitrile of Formula (G) in an organic solvent in presence of a base to obtain crude vilazodone free base; 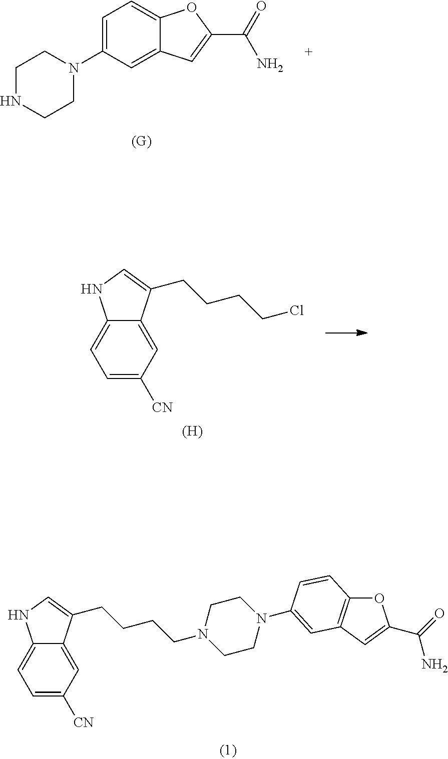
ii) purifying the crude vilazodone free base of step (i) in an organic solvent;
iii) treating the purified vilazodone free base of step (ii) with an organic solvent to obtain solid state form-Z of vilazodone of Formula (1).

7. The process as claimed in claim 6(i), wherein organic solvent comprises one or more of toluene, xylene, ethylbenzene dimethyl formamide, dimethyl sulfoxide, dimethyl acetamide, acetonitrile, C₁-C₄ straight chain or branched alcohols, acetone, methyl isobutyl ketone, methyl ethyl ketone, ethyl acetate, isopropyl acetate and butyl acetate or mixture thereof.

8. The process as claimed in claim 7, wherein organic solvent may be dimethyl sulfoxide.

9. The process as claimed in claim 6(i), wherein base comprises of organic or inorganic base; selected from diisopropylethylamine, diisopropylamine, trimethylamine, diethylamine, piperidine, morpholine, pyridine, DBU, DABCO, an alkali and alkaline metal hydroxide and carbonate like sodium hydroxide, potassium hydroxide, lithium hydroxide sodium carbonate, potassium carbonate and cesium carbonate.

10. The process as claimed in claim 9, wherein base may be diisopropylethylamine.

11. The process as claimed in claim 6(ii), wherein organic solvent may be acetone. 10

12. The process as claimed in claim 6(iii), wherein organic solvent may be methanol.

13. The process as claimed in claim 6(iii), wherein acid used may be an organic and mineral acid.

14. The process as claimed in claim 13, wherein acid may be acetic acid.

15. Use of solid state Form-Z of vilazodone of Formula (1) in the preparation of vilazodone hydrochloride.

16. A process for preparing vilazodone of Formula (1) having particle size in terms of d95 is less than about 10 microns;
the process comprising:
i) milling vilazodone having particle size in terms of d95 is greater than about 100 microns;
ii) slurrying micronized vilazodone in one or more organic solvents to form a solution;
iii) isolating vilazodone having particle size in terms of d95 is less than about 10 microns.

17. The process as claimed in claim 16, wherein milling is performed with a feeding pressure of about 3 kg and grinding pressure of 4 kg.

18. The process as claimed in claim 16, wherein solvent comprises is selected from one or more of C₃-C₆ ketones, water, N-methylpyrrolidone, C₃-C₆ amides, halosubstituted C₆-C₁₂ aromatic hydrocarbons, propylene glycol, diemthyl sulfoxide, diemthyl carbonate, C1-8 alkyl alcohols, acetonitrile, C₂-C₆ alkyl acetates, cellosolve, dimethyl carbonate, polyethylene glycol methyl ether and C₂-C₈ ethers.

19. A pharmaceutical composition comprising a therapeutically effective amount of an amorphous form of vilazodone hydrochloride and one or more pharmaceutically acceptable carriers, excipients, or diluents.

20. A pharmaceutical composition comprising a therapeutically effective amount of solid state Form-Z of vilazodone having particle size in terms of d95 less than about 100 microns and one or more pharmaceutically acceptable carriers, excipients, or diluents.

21. Use of solid state Form-Z of vilazodone for the treatment of major depressive disorders.

22. A method of treatment of major depressive disorders comprising administering a suitable dose of solid state Form-Z of vilazodone to a subject.
